(19) United States
(12) Patent Application Publication
(10) Pub. No.: US 2003/0219802 A1
(43) Pub. Date: Nov. 27, 2003

(54) Diagnosis and treatment of osteosarcoma

(75) Inventors: Hassan R. Dhaini, Ann Arbor, MI (US); Laurence H. Baker, Ann Arbor, MI (US); Paul F. Hollenberg, Ann Arbor, MI (US); Timothy D. Johnson, Ann Arbor, MI (US); Dafydd G. Thomas, Fenton, MI (US)

Correspondence Address: MEDLEN & CARROLL, LLP, Suite 350, 101 Howard Street, San Francisco, CA, 94105, US

(73) Assignee: The Regents of the University of Michigan, Ann Arbor, MI

(21) Appl. No.: 10/384,016

(22) Filed: Mar. 7, 2003

Related U.S. Application Data
(60) Provisional application No. 60/362,951, filed on Mar. 8, 2002.

(51) Int. Cl. C12Q 1/68; G01N 33/574

(52) U.S. Cl. 435/6; 435/7.23

(57) ABSTRACT

The present invention relates to compositions and methods for the diagnosis, prognosis, and treatment of cancer. In particular, the present invention provides compositions and methods of using P450 3A4/5 expression in the diagnosis, prognosis, and treatment of osteosarcoma. The present invention thus provides improved compositions and methods for providing prognoses to osteosarcoma patients.

This application claims priority to Provisional patent application serial No. 60/362,951, filed Mar. 8, 2002.

### FIELD OF THE INVENTION

The present invention relates to compositions and methods for the diagnosis, prognosis, and treatment of cancer. In particular, the present invention provides compositions and methods of using P450 3A4/5 expression in the diagnosis, prognosis, and treatment of osteosarcoma.

### BACKGROUND OF THE INVENTION

Most forms of cancer do not have diagnostic screening tests available. For the cancers that do have screening tests available, the tests are frequently invasive, expensive, and lack strong diagnostic and prognostic utility.

For example, osteosarcoma is the most frequent primary malignant bone tumor, mainly occurring in children and adolescents. It accounts for approximately 30% of all primary bone tumors of the skeleton (Chindia et al., Oral Oncol., 37:545 [2001]). Osteosarcoma occurs most commonly in the metaphysis of the long bones, distal and proximal femur and the proximal humerus. The natural history of osteosarcoma is characterized by a rapidly progressive course with early metastases primarily to the lungs, which generally occurs within 1-2 years despite amputation. Pulmonary metastasis portends a poor prognosis. Before chemotherapy became routinely used about 90% of patients recurred within 2 years of diagnosis in spite of aggressive surgical treatments (Sluga et al., Clin. Orthop. Rel. Res., 358:120 [1999]). As a result of the introduction of adjunctive chemotherapy 20 years ago, an improvement in long-term survival rate from 10-20% to nearly 50-80% can be achieved (Winkler et al., J. Clin. Oncol., 2:617 [1984]). Although prognosis increased dramatically, drug resistance and poor clinical outcome are still major problems in the treatment of these tumors (Scotlandi et al., Cancer Res., 56:2434 [1996]).

Thus far, a reliable biomarker predicting clinical outcome at diagnosis does not exist. To date, the most useful determinant of which patients develop metastatic lesions is pathological review of chemotherapy-induced necrosis. Clearly there is a need to determine which patients will benefit from more aggressive pre-operative therapy or perhaps require less aggressive chemotherapy. Therefore, the identification of new prognostic markers is extremely important.

### SUMMARY OF THE INVENTION

The present invention relates to compositions and methods for the diagnosis prognosis, and treatment of cancer. In particular, the present invention provides compositions and methods of using P450 3A4/5 expression in the diagnosis, prognosis and treatment of osteosarcoma.

For example, in some embodiments, the present invention provides a method for characterizing tumor tissue in a subject, comprising: providing tumor tissue from a subject; and detecting the presence or absence of P450 3A4/5 in the tumor tissue, thereby characterizing the tumor tissue. In some embodiments, the tumor tissue is osteosarcoma tumor tissue. In some embodiments, the tumor tissue is biopsy tissue. In other embodiments, the tumor tissue is post-surgical tumor tissue. In some embodiments, detecting P450 3A 4/5 comprises detecting the presence of P450 3A 4/5 mRNA. In some embodiments, detecting the presence of P450 3A 4/5 mRNA comprises exposing the P450 3A 4/5 mRNA to a nucleic acid probe complementary to at least a portion of the P450 3A 4/5 mRNA. In some embodiments, detecting the presence of P450 3A 4/5 mRNA comprises a detection assay selected from the group consisting of a Northern blot, in situ hybridization, reverse-transcriptase polymerase chain reaction, and microarray analysis. In other embodiments, detecting the presence of P450 3A 4/5 comprises detecting the presence of a P450 3A 4/5 polypeptide. In some embodiments, detecting the presence of a P450 3A 4/5 polypeptide comprises exposing the P450 3A 4/5 polypeptide to an antibody that specifically binds to P450 3A 4/5 and detecting the binding of the antibody to the P450 3A 4/5 polypeptide. In some embodiments, the detecting comprises immunocytochemistry. In some preferred embodiments, the immunocytochemistry is quantitative immunocytochemistry. In some embodiments, characterizing comprises identifying the risk of the tumor tissue metastasizing based on the detecting the presence of P450 3A 4/5.

The present invention also provides a method of determining a treatment course of action, comprising: providing tumor tissue from a subject suffering from cancer; and detecting the presence or absence of P450 3A4/5 in the tumor tissue; and determining a treatment course of action based on the presence or absence of P450 3A4/5 in the tumor tissue. In some embodiments, the presence of P450 3A4/5 in the tumor tissue result is the treatment course of action being aggressive treatment. In some embodiments, the aggressive treatment comprises chemotherapy. In some embodiments, the chemotherapy is administered prior to surgical removal of the cancer from the subject. In other embodiments, the absence of P450 3A4/5 in the tumor tissue result is the treatment course of action being non-aggressive treatment. In some embodiments, the tumor tissue is osteosarcoma tumor tissue. In some embodiments, the tumor tissue is biopsy tissue. In other embodiments, the tumor tissue is post-surgical tumor tissue. In some embodiments, detecting P450 3A 4/5 comprises detecting the presence of P450 3A 4/5 mRNA. In other embodiments, detecting the presence of P450 3A 4/5 comprises detecting the presence of a P450 3A 4/5 polypeptide. In some embodiments, detecting the presence of a P450 3A 4/5 polypeptide comprises exposing the P450 3A 4/5 polypeptide to an antibody that specifically binds to P450 3A 4/5 and detecting the binding of the antibody to the P450 3A 4/5 polypeptide. In some embodiments, the detecting comprises immunocytochemistry. In some preferred embodiments, the immunocytochemistry is quantitative immunocytochemistry.

The present invention further provides a kit for characterizing cancer in a subject, comprising a reagent that specifically detects the presence of absence of expression of P450 3A 4/5; and instructions for using the kit for characterizing cancer in the subject. In some embodiments, the reagent comprises an antibody that specifically binds to P450 3A 4/5. In other embodiments, the reagent comprises a nucleic acid probe that specifically binds to a P450 3A 4/5 mRNA. In some embodiments, the instructions comprise instructions required by the United States Food and Drug Administration for use in in vitro diagnostic products.

The present invention additionally provides a method of quantitating protein expression in a tissue sample, comprising providing a tissue microarray comprising at least one protein of interest; reagents capable of specifically detecting each of the at least one proteins of interest; and a fluorescence microscopy apparatus; and treating the tissue microarray with the reagents and the fluorescence microscopy apparatus to generate digitized images corresponding to each of the at least one protein of interest; and calculating a nuclear density weighted average enzyme pixel intensity of the digitized images corresponding to each of the at least one protein of interest. In some embodiments, the tissue microarry is derived from tumor biopsy samples. In some embodiments, the tumor biopsy samples are osteosarcoma samples. In some embodiments, the protein of interest is P450 3A4/5. In some embodiments, the reagents comprise antibodies specific for each of the at least one protein of interest.

In still further embodiments, the present invention a method of screening compounds, comprising providing a cell sample comprising cancer cells; and one or more test compounds; and contacting the sample with the test compound; and detecting an increase or decrease in P450 3A4/5 expression in the sample in the presence of the test compound relative to the absence of the test compound. In some embodiments, the cancer cells are osteosarcoma cells. In some embodiments, contacting the sample with the test compound results in death of the cancer cells. In some embodiments, contacting the sample with the test compound results in reduced growth of the cancer cells. In some embodiments, detecting comprises detecting P450 3A4/5 mRNA. In other embodiments, detecting comprises detecting P450 3A4/5 polypeptide. In some embodiments, the cell is in vitro. In other embodiments, the cell is in vivo. In some embodiments, the test compound comprises an antisense compound. In some embodiments, the test compound comprises a drug. In some embodiments, the drug is an antibody. In some embodiments, the drug specifically binds to P450 3A4/5.

In yet other embodiments, the present invention provides a method of screening compounds, comprising providing an cell sample expressing P450 3A4/5; and one or more test compounds; and contacting the sample with the test compound; and detecting a change in viability of the cell sample in the presence of the test compound relative to the absence of the test compound. In some embodiments, the test compound is a cancer chemotherapeutic. In some embodiments, the test compound is a candidate cancer therapeutic. In some embodiments, the cell sample is a cancerous cell sample. In some embodiments, the cancerous cell sample is a osteosarcoma sample. In some embodiments, the cell is in vitro. In other embodiments, the cell is in vivo.

### DESCRIPTION OF THE FIGURE

FIG. 1 shows P450 3A4/5 levels described as weighted average pixel intensity in the total osteosarcoma population.

### GENERAL DESCRIPTION OF THE INVENTION

The present invention provides novel markers for cancers (e.g., osteosarcomas) that are likely to metastasize. Several proteins expressed in osteosarcomas and present in serum levels have been proposed as prognostic factors. Expression of p53 and its mutants has been reported in many human malignancies and was reported as a prognostic marker in some types of cancer (Marx et al., Euro J Cancer 34:845 [1998]; Schimtz-Drager et al., European Urology 38:691 [2000]). However, several studies did not find any correlation between its expression and prognosis or response to chemotherapy, suggesting that p53 is not a marker for osteosarcomas (Miller et al., J Cancer Res Clin Oncol 122:559 [1996]; Yokoyama et al., Pathol Res Pract 194:615 [1998]). Overexpression of Her2 and poor outcome has been described for breast cancer. Although it was shown to correlate with poor prognosis and early pulmonary metastases in human osteosarcoma (Onda et al., Cancer 77:71 [1996]) recent reports show absence of Her2 in osteogenic sarcomas. In addition, metallothionein expression did not show any correlation with outcome (Uozaki et al., Cancer 79:2336 [1997]) and MDR-1 which, despite studies that suggest a role for p-glycoprotein in resistance to doxorubicin (Baldini et al., J Orthop Res 17:629 [1999]) still present contradicting evidence on correlation between its expression and preoperative chemotherapy (Wunder et al., J Clin Oncol 18:2685 [2000]; Nanni et al., Oncogene 18:739 [1999]). As for serological markers, reports on the value of lactate dehydrogenase and alkaline phosphatase as prognostic factors are still controversial (Bacci et al., J Chemther 1996; 8:472 [1996] Pochgool et al., Clin Orthop Rel Res 345:206 [1997]; Meyers et al., J Clin Oncol 11:449 [1993]).

A recent study in a large cohort of osteosarcoma patients defined several independent prognostic factors (Bielack et al., J Clin Oncol 20:776 [2002]). Although some of these, such as tumor site and size, are assessable at diagnosis, a reliable prediction of prognosis is not possible until later in the course of the disease, when information on tumor response and the quality of surgical remission become available.

The cytochrome P450 dependent mixed-function oxidases are heme-containing proteins that play an important role in cell regulation as a consequence of their involvement in the metabolism of a wide variety of endogenous compounds active in cellular signaling, including steroids, fatty acids, and eicosanoids, with many of the metabolites formed by the P450s having been implicated in multiple steps of tumorigenesis and metastasis (Felder et al., J. Pharmacol. Expl. Ther., 267:967 [1991]; Butcher et al., Cancer Res., 53:3405 [1993]). Moreover, cytochrome P450s are thought to mediate invasion and metastasis by generation of reactive oxygen species (ROS) during various metabolic steps, and by participating in the activation of protein kinases, cellular proteoglycan changes and lysosomal enzymes (Parke et al., EHP 102:852 [1994]). P450s are involved in the activation and detoxification of a large number of anti-cancer drugs, many of which are used to treat ostesarcomas (etoposide, ifosfamide and adriamycin), with some isoenzymes showing tumor specific expression (Murray et al., Gut 35:599 [1994]). Therefore, P450 enzymes play a role both in carcinogenesis and in influencing the metabolism of chemotherapeutic agents used in sarcoma treatment. The present invention is not limited to a particular mechanism. Indeed, an understanding of the invention is not necessary to practice the present invention. Nonetheless, it is contemplated that P450 enzymes play important roles in both the occurrence and the treatment of the tumors. To date no one has investigated the expression of cytochrome P450 enzymes in osteosarcomas.

Individual sub-families of the cytochromes P450 have been reported to be present in several varieties of sarcomas and carcinomas (Murray et al., J. Pathol., 169:347 [1993]; Forrester et al., Carcinogenesis 11:2163 [1990]). P450 3A4/5, which is considered to be the most clinically relevant P450 in view of its broad specificity, and since it is the primary cytochrome P450 found in the liver accounting for more than 25% of total liver cytochromes P450 (Watkins et al., PNAS 82:6310 [1985]), is involved in the metabolism of various anti-cancer drugs (Berthou et al., 47:1883 [1994]).

P450 3A4 is of particular interest since it has previously been shown to be expressed in high frequency in sarcomas (Massaad et al., Cancer Res.,52:6567 [1992]) and is involved in the oxidation of ifosfamide, vinblastine, etoposide and doxorubicin (Zhou-Pan et al., Cancer Res 53:5121 [1993]; Zhao et al., Drug Metab Dispos 26:188 [1998]; Michalets, Pharmacotherapy 18:84 [1998]; Brain et al., Br J Cancer 77:1768 [1998]; Weber and Waxman, Biochemical Pharmacology 45:1685 [1993]), all four types of compounds used as chemotherapeutic agents for the treatment of osteosarcomas.

Experiments conducted during the course of the development of the present invention examined the expression of five major cytochrome P450 isoenzymes in a cohort of osteosarcoma primary biopsies by regular immunocytochemistry. The present invention further provides a novel quantitative imunocytochemistry technique (QICC) to assess the levels of P450 3A4/5 in osteosarcoma tissue sections. The present invention thus provides novel methods of providing cancer prognoses and treatments.

Definitions

To facilitate an understanding of the present invention, a number of terms and phrases are defined below:

As used herein, the term “P450 3A 4/5” refers to either P450 3A4 or P450 3A5 polypeptides or functional variants therof (e.g., polypeptides that differ by one or more amino acids from wild type P450 3A4 or P450 3A5 but that retain the biological activities of P450 3A4 or P450 3A5).

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F (ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject diagnosed with a cancer” refers to a subject who has been tested and found to have cancerous cells. The cancer may be diagnosed using any suitable method, including but not limited to, biopsy, x-ray, CT scan, MRI, and blood test.

As used herein, the term “initial diagnosis” refers to a test result of initial cancer diagnosis that reveals the presence or absence of cancerous cells (e.g., using a biopsy and histology). An initial diagnosis does not include information about the stage of the cancer or the risk of metastasis.

As used herein, the term “post surgical tumor tissue” refers to cancerous tissue (e.g., osteosarcoma) that has been removed from a subject (e.g., during surgery).

As used herein, the term “identifying the risk of said tumor metastasizing” refers to the relative risk (e.g., the percent chance or a relative score) of a tumor (e.g., an osteosarcoma) metastasizing.

As used herein, the term “subject at risk for cancer” refers to a subject with one or more risk factors for developing a specific cancer. Risk factors include, but are not limited to, gender, age, genetic predisposition, environmental expose, and previous incidents of cancer, preexisting non-cancer diseases, and lifestyle.

As used herein, the term “characterizing cancer in subject” and “characterizing tumor tissue in a subject” refer to the identification of one or more properties of a cancer or tumor sample in a subject, including but not limited to, the presence of benign, pre-cancerous or cancerous tissue and the stage of the cancer. Cancers may be characterized by the identification of P450 3A 4/5 expression in tumor tissues.

As used herein, the term “providing tumor tissue from a subject” refers to both tumor tissue excised from a subject (e.g., biopsy tissue) and tumor tissue in vivo. In some embodiments, tumor tissue is provided in the form of a biopsy sample preserved and affixed to a solid support (e.g., a microscope slide). In other embodiments, tumor tissues are provided as formalin-fixed, paraffin-embedded blocks.

As used herein, the term “treatment course of action” refers to all of the care given to a subject (e.g., a subject with cancer) including, but not limited to, surgery, medication (e.g., chemotherapy), and radiation treatment. In some embodiments, the presence or absence of P450 3A 4/5 is a tumor is used in the choice of treatment course of action.

As used herein, the term “aggressive treatment” refers to a treatment course of action that provides aggressive choices of medication (e.g., chemotherapy) and surgery. Aggressive treatment is given on an accelerated time course (e.g., as soon after the treatment course of action is chosen as possible). In contrast, “non-aggressive” treatment provides reduced or less aggressive choices of medication.

As used herein, the term “tissue micorarray” refers to a solid surface comprising a plurality of addressed tissue samples. The location of each of the samples in the microarray is known, so as to allow for identification of the samples following analysis. In some embodiments, tissue microarrays are generated from biopsy samples (See e.g., Example 1 below).

As used herein, the term “reagent(s) capable of specifically detecting P450 3A 4/5 expression” refers to reagents used to detect the expression of P450 3A 4/5. Examples of suitable reagents include, but are not limited to, nucleic acid probes capable of specifically hybridizing to P450 3A 4/5 mRNA or cDNA, and antibodies.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination. Additional information is available at the Internet web page of the U.S. FDA.

As used herein, the term “non-human transgenic animal lacking a functional P450 3A 4/5 gene” refers to a non-human animal (preferable a mammal, more preferably a mouse) whose endogenous P450 3A 4/5 gene has been inactivated (e.g., as the result of a “P450 3A 4/5 knockout” or a “P450 3A 4/5 knock-in”).

As used herein, the terms “P450 3A 4/5 knockout” refers to a non-human animal (e.g., a mouse) lacking a functional P450 3A 4/5 gene. In some embodiments, the entire P450 3A 4/5 gene is deleted. In other embodiments, the gene is inactivated via other means (e.g., deletion of essential portions or inversions of some or all of the P450 3A 4/5 gene). In other embodiments, the P450 3A 4/5 gene is inactivated using antisense inhibition. P450 3A 4/5 knockouts include conditional knockouts (e.g., selective inhibition of gene activity). P450 3A 4/5 knockout mice may be made using any suitable method including, but not limited to, those described herein. P450 3A 4/5 genes can also be inactivated via the construction of a “P450 3A 4/5 knock-in” in which the gene is inactivated by the insertion of exogenous DNA into a region of the gene required for function.

As used herein, the term “detecting a decrease in viability” refers to a decrease in the number of living cells in a culture.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and preferably, the risk of metastasis).

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample. “Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA or protein to indicate a level of expression approximately 2-fold higher (or greater) than that observed in a given tissue in a control. Levels of mRNA or protein are measured using any of a number of techniques known to those skilled in the art including, but not limited to Northern blot analysis and the quantitative immunofluorescence technique of the present invention (See e.g., Example 3). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]), has been modified by several groups to optimize conditions for particular types of cells. The art is well aware of these numerous modifications.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanie phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to compositions and methods for the diagnosis, prognosis, and treatment of cancer. In particular, the present invention provides compositions and methods of using P450 3A4/5 expression in the diagnosis, prognosis and treatment of osteosarcoma.

I. P450 3A4/5 as a Marker for Osteosarcoma Metastasis

The present invention relates to compositions and methods for cancer diagnostics, including but not limited to, P450 3A4/5 cancer markers. In particular, the present invention provides markers (e.g., P450 3A4/5) whose expression in tumors is specifically correlated with tumors that are likely to metastasize. Such markers find use in the characterization of cancer in subjects (e.g., humans or other animals).

A. Identification of Markers

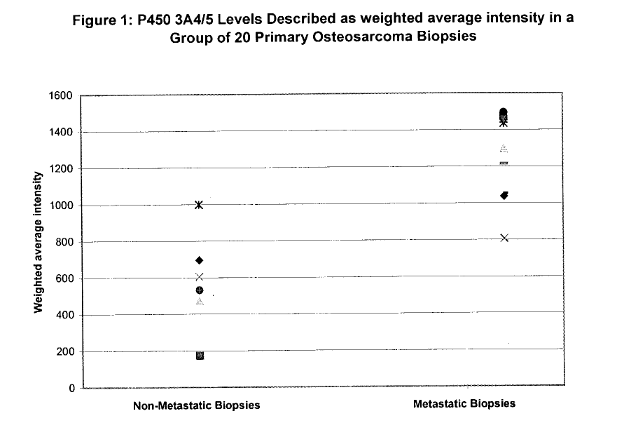

Experiments conducted during the development of the present invention resulted in the finding that P450 3A4/5 expression in tumor cells is correlated with metastatic tumors. Primary osteosarcoma biopsies using tissue microarray blocks were analyzed for the expression of 5 major cytochrome P450 isoenzymes using routine immunocytochemical methods. Results of experiments conducted during the course of development of the present invention showed 3A4/5 to have a high frequency of occurrence with noticeable variation in the levels observed. P450 3A4/5 was found to have higher expression in metastatic primary tumors, independent of other prognostic tests such as the Huvos grade and chemotherapy-induced necrosis of the tumors (See Table 1 and FIG. 1).

Thus, the present invention provides methods of predicting tumor (e.g., osteosarcoma) metastasis. Such information allows for the determination of clinical courses of action. For example, patients whose tumors are more likely to metastasize may elect to start aggressive chemotherapy or experimental treatments at an earlier time. Alternatively, some patients whose tumors are likely to metastasize may elect to forgo unpleasant and invasive treatment such as chemotherapy.

The present invention is not limited to the characterization and treatment of osteosarcomas. It is contemplated that P450 3A4/5 is over expressed in other types of cancer and thus has diagnostic use in all tumors in which P450 3A4/5 expression is correlated with tumor diagnosis or progression. Additional tumors having an association with P450 3A4/5 expression can be identified using any suitable method including, but not limited to, the methods of the present invention (See e.g., Example 3).

C. Detection of P450 3A4/5

In some embodiments, the present invention provides methods for detection of P450 3A4/5. In preferred embodiments, the presence of P450 3A4/5 protein or mRNA is measured directly. In some embodiments, P450 3A4/5 mRNA or protein is detected in tissue samples (e.g., biopsy samples). In other embodiments, P450 3A4/5 mRNA or protein is detected in bodily fluids (e.g., serum, plasma, or urine). The present invention further provides kits for the detection of P450 3A4/5. In preferred embodiments, the presence of P450 3A4/5 is used to provide a diagnosis or prognosis to a subject.

In some preferred embodiments, P450 3A4/5 protein is detected. Protein expression may be detected by any suitable method. In some embodiments, proteins are detected by binding of an antibody specific for the protein. In some preferred embodiments, antibody binding is detected and quantitated using the novel quantitative immunofluorescence methods of the present invention (See Example 3). The quantitative immunofluorescence method described in Example 3 comprises the measurement of nuclei and P450 3A4/5 immunofluorescence, followed by the partitioning of the image into equal-sized areas. The nuclear-density-weighted average enzyme pixel intensity is then computed for each area. The quantitative immunofluorescence method of the present invention is not limited to the detection of P450 3A 4/5. It is suitable for the quantitation of any immunofluorescence image of a tissue (e.g., additional tumor markers). Additional tumor markers include, but are not limited to, those disclosed in Int J Biochem Cell Biol.,33:11-7 [2001]; Clin Orthop.(382):59-65 [2001] (Human epidermal growth factor receptor 2); J Clin Oncol. Feb. 1, 2002;20(3):776-90; J Bone Joint Surg Am. January 2002;84-A(1):49-57; Oncol Rep. January-February 2002;9(1):171-5; Cancer. 79(12):2336-44, Jun. 15, 1997.

The present invention is not limited to any particular method of detecting protein expression described. Any suitable method may be utilized. For example, in some embodiments, antibody binding is detected using a suitable technique, including but not limited to, radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include, but are not limited to, those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a diagnosis and/or prognosis based on the presence or absence of a series of proteins corresponding to cancer markers is utilized.

In other embodiments, the immunoassay described in U.S. Pat. Nos. 5,599,677 and 5,672,480, each of which is herein incorporated by reference, is utilized. In other embodiments, proteins are detected by immunohistochemistry.

In other embodiments, P450 3A4/5 is detected at the level of P450 3A4/5 RNA. In some embodiments, P450 3A4/5 RNA is detected by measuring the expression of corresponding mRNA in a tissue sample (e.g., prostate or colon tissue). mRNA expression may be measured by any suitable method, including but not limited to, those disclosed below.

In some embodiments, RNA is detected by Northern blot analysis. Northern blot analysis involves the separation of RNA and hybridization of a complementary labeled probe. Methods for Northern blot analysis are well known in the art.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to an oligonucleotide probe. A variety of hybridization assays using a variety of technologies for hybridization and detection are available. For example, in some embodiments, TaqMan assay (Applied Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

D. Kits

In some embodiments, the present invention provides kits for the characterization of cancer (e.g.,osteosarcoma). In some embodiments, the kits contain antibodies specific for P450 3A4/5, in addition to detection reagents and buffers. In other embodiments, the kits contain reagents specific for the detection of P450 3A4/5 mRNA or cDNA (e.g., oligonucleotide probes or primers). In preferred embodiments, the kits contain all of the components necessary to perform a detection assay, including all controls, directions for performing assays, and any necessary software for analysis and presentation of results. In some embodiments, the kits contain instructions include a statement of intended use as required by the U.S. Food and Drug Administration for the labeling of in vitro diagnostic assays (See above description of what is required in such a statement).

II. Antibodies

The present invention provides isolated antibodies. In preferred embodiments, the present invention provides monoclonal antibodies that specifically bind to an isolated polypeptide comprised of at least five amino acid residues of P450 3A4/5. These antibodies find use in the diagnostic and therapeutic methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of mycloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against P450 3A4/5). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against P450 3A4/5) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, P450 3A4/5 protein (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

In some embodiments, antibodies (e.g., monoclonal antibodies) are humanized. Such humanized antibodies find particular use in the cancer immunotherapies described below. Humanized antibodies are altered in order to make them less immunogenic to humans, e.g., by constructing chimeric antibodies in which a mouse antigen-binding variable domain is coupled to a human constant domain. Humanized antibodies are typically human antibodies in which some CDR residues and possibly some FR residues are substituted by residues from analogous sites in rodent antibodies. Methods for humanizing antibodies are well known in the art and include but are not limited to, those disclosed in U.S. Pat. Nos. 6,054,297, 4,816,567, 6,180,377, 5,871,907, 5,585,089, and 6,180,370, each of which is herein incorporated by reference.

III. Drug Screening

In some embodiments, the present invention provides drug screening assays (e.g., to screen for anticancer drugs). The present invention is not limited to a particular mechanism. Indeed, and understanding of the mechanism is not necessary to practice the present invention. Nonetheless, it is contemplated that P450 3A4/5 overexpression in malignancies with metastatic potential may be a cellular protective mechanism conferring survival advantage by providing these tumors with the metabolic mechanisms for the inactivation of chemotherapeutic agents. It is also contemplated that P450 3A4/5 may be involved in growth regulation through metabolism of endogenous growth regulatory factors. Accordingly, the present invention provides drugs screening methods for identifying compounds that alter (e.g., decrease) the expression of P450 3A4/5 (e.g., in tumor tissue). The present invention further provides methods of identifying chemotherapeutic agents that are active in P450 3A 4/5 expressing cancers. In some embodiments, candidate compounds are antisense agents (e.g., oligonucleotides) directed against P450 3A4/5. See Section IV below for a discussion of antisense therapy. In other embodiments, candidate compounds are antibodies (e.g., those described in Section II above). In other embodiments, candidate compounds are small molecules.

A. P450 3A4/5 Expression Assays

In one screening method, candidate compounds are evaluated for their ability to alter P450 3A4/5 expression by contacting a compound with a cell expressing P450 3A4/5 and then assaying for the effect of the candidate compounds on expression. In some embodiments, the effect of candidate compounds on expression of P450 3A4/5 is assayed for by detecting the level of P450 3A4/5 mRNA expressed by the cell. mRNA expression can be detected by any suitable method, including but not limited to, those disclosed herein.

In other embodiments, the effect of candidate compounds is assayed by measuring the level of P450 3A4/5 polypeptide expression. The level of polypeptide expressed can be measured using any suitable method, including but not limited to, those disclosed herein.

In other embodiments, the effect of candidate compounds on the viability of cells is measured. For example, in some embodiments, the rate of cell division or growth in the presence and absence of candidate compounds is measured.

B. Screening for Chemotherapeutic Agents that Alter Viability

In some embodiments, the present invention provides methods of identifying chemotherapeutic agents that are active in P450 3A 4/5 expressing tumors. In some embodiments, the chemotherapeutic agents are known cancer chemotherapeutic agents (See e.g., Section IV below). In other embodiments, the agents are candidate chemotherapeutic agents that have not yet been identified as cancer therapeutics.

The effect of candidate or known chemotherapeutics on P450 3A 4/5 expressing tumors can be identified using any suitable assay. For example, in some embodiments, cells (e.g., cancer cells) are engineered or selected to express P450 3A 4/5. These cells are then contacted with the chemotherapeutic agents and the viability of the cells is assessed. For example, in some embodiments, the rate of cell division or growth in the presence and absence of candidate compounds is measured. Preferred chemotherapeutic agents are those that decrease the viability of the cells (e.g., inhibit growth of cells or kill cells).

C. In Vitro Assays

In some embodiments, in vitro drug screens are performed using purified P450 3A4/5. In some embodiments, the P450 3A4/5 proteins are immobilized to facilitate separation of complexed from uncomplexed forms of one or both of the proteins, as well as to accommodate automation of the assay. Binding of a test compound to P450 3A4/5 can be accomplished in any vessel suitable for containing the reactants. Examples of such vessels include microtitre plates, test tubes, and microcentrifuge tubes. In one embodiment, a fusion protein can be provided which adds a domain that allows proteins to be bound to a matrix. For example, glutathione-S-transferase/AIP-6 fusion proteins or glutathione-S-transferase/target fusion proteins can be adsorbed onto glutathione sepharose beads (Sigma Chemical; St. Louis, Mo.) or glutathione derivatized microtitre plates, which are then combined with the test compound or the test compound and the non-adsorbed protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly. Alternatively, the complexes can be dissociated from the matrix, and the level of protein binding or activity determined using standard techniques.

Other techniques for immobilizing proteins on matrices can also be used in the screening assays of the invention. For example, P450 3A4/5 can be immobilized utilizing conjugation of biotin and streptavidin. Biotinylated P450 3A4/5 can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with P450 3A4/5 but which do not interfere with binding of the protein to test compounds can be derivatized to the wells of the plate, and unbound protein trapped in the wells by antibody conjugation. Methods for detecting such complexes, in addition to those described above for the GST-immobilized complexes, include immunodetection of complexes using antibodies reactive with P450 3A4/5, as well as enzyme-linked assays that rely on detecting an enzymatic activity associated with the P450 3A4/5.

In other embodiments, a competitive drug screening assays in which neutralizing antibodies capable of binding P450 3A4/5 specifically compete with a test compound for binding P450 3A4/5 are utilized. In this manner, the antibodies can be used to detect the presence of any compound that shares one or more antigenic determinants with P450 3A4/5.

D. In Vivo Assays

In still further embodiments, transgenic animals having altered (e.g., inactivated or overexpressed) P450 3A4/5 gene are utilized in drug screening applications. For example, in some embodiments, transgenic animals that overexpress P450 3A4/5 in tissues that typically have P450 3A4/5+ tumors (e.g., bone) are utilized for drug screening. Such mice are administered libraries of compounds and a decrease in tumor size, lack of tumor metastasis or lack of expression of P450 3A4/5 is screened for.

IV. Cancer Therapies

In some embodiments, the present invention provides therapies for cancer (e.g., osteosarcoma). In some embodiments, therapies target P450 3A4/5.

A. Antisense Therapies

In some embodiments, the present invention targets the expression of P450 3A4/5. For example, in some embodiments, the present invention employs compositions comprising oligomeric antisense compounds, particularly oligonucleotides (e.g., those identified in the drug screening methods described above), for use in modulating the function of nucleic acid molecules encoding P450 3A4/5, ultimately modulating the amount of P450 3A4/5 expressed. This is accomplished by providing antisense compounds that specifically hybridize with one or more nucleic acids encoding P450 3A4/5. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of P450 3A4/5. In the context of the present invention, “modulation” means either an increase (stimulation) or a decrease (inhibition) in the expression of a gene. For example, expression may be inhibited to potentially prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding P450 3A4/5. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the tenns “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

B. Genetic Therapies

The present invention contemplates the use of any genetic manipulation for use in modulating the expression of P450 3A4/5. Examples of genetic manipulation include, but are not limited to, gene knockout (e.g., removing the P450 3A4/5 from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

C. Antibody Therapy

In some embodiments, the present invention provides antibodies that target P450 3A4/5 expressing tumors. In preferred embodiments, the antibodies used for cancer therapy are humanized antibodies.

In some embodiments, the therapeutic antibodies comprise an antibody generated against P450 3A4/5, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeted P450 3A4/5. Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

D. Small Molecule Drugs

In some embodiments, the present invention provides drugs (e.g., small molecule drugs) that reduce or eliminate cancer by inhibiting the biological activity of P450 3A4/5. In some embodiments, small molecule drugs are identified using the drug screening methods described above. In preferred embodiments, the small molecule drugs of the present invention result in an increased rate of death of cancer cells than normal cells. In some embodiments, small molecule drugs are identified using the drug screens described herein (e.g., in Section III above).

E. Pharmaceutical Compositions

The present invention further provides pharmaceutical compositions (e.g., comprising the therapeutic compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense compounds and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

V. Transgenic Animals Having Altered P450 3A4/5

The present invention additionally contemplates the generation of transgenic animals comprising an exogenous P450 3A4/5 gene or mutants and variants thereof (e.g., truncations, deletions, insertions, or single nucleotide polymorphisms). In other embodiments, the present invention provides transgenic animals with a knock-out of the P450 3A4/5 gene. In still further embodiments, transgenic animals overexpress P450 3A4/5 in specific tissues (e.g., bone). In yet other embodiments, transgenic animals having altered P450 3A4/5 genes are crossed with other cancer models to general animals with multiple transgenes. In preferred embodiments, the transgenic animal displays an altered phenotype (e.g., increased or decreased presence of P450 3A4/5) as compared to wild-type animals. Methods for analyzing the presence or absence of such phenotypes include but are not limited to, those disclosed herein. In some preferred embodiments, the transgenic animals further display an increased or decreased growth of tumors or evidence of metastatic cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized knock-out gene function or create deletion mutants (e.g., truncation mutants). Methods for homologous recombination are described in U.S. Pat. No. 5,614,396, incorporated herein by reference.

Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); and ° C. (degrees Centigrade).

### EXAMPLE 1

### Methods

Patients

Formalin-fixed, paraffin-embedded blocks originally derived from primary bone tumors were obtained from the files of the Department of Pathology, University of Michigan Medical Center, Ann Arbor, Mich. The diagnosis was confirmed and IRB approval was obtained.

The osteosarcoma cases (n=18) had an age range of 6-29 years, with a mean age of 14.6 years and a male:female ratio of 10:8. All eighteen cases were primary biopsies with 11 tumors that metastasized to the lung, and 7 tumors with no record of metastases. There were 9 osteosarcomas from the femur, 8 from the tibia, and 1 from the humerus.

Immunocytochemical Staining for Cytochromes P450

Initially, tissue micro array blocks containing 18 biopsies and their corresponding resections were assembled as described (Kononen et al., Nature Medicine 4:844 [1998]). Then these were sectioned, de-paraffinized, and stained as follows: 5 μm sections were microwave-preheated in citric acid buffer to retrieve antigenicity. Sections were incubated with blocking solution for 60 min at room temperature prior to being exposed to the primary antibody of P450s 1A1/2 (Goat polyclonal antibody, cat# 299124, Gentest, Woburn, Mass., 1:500), 1B1 (Rabbit polyclonal antibody, cat# A211, Gentest, Woburn, Mass., 1:500), 2B6 (Rabbit polyclonal antibody, cat# A226, Gentest, Woburn, Mass., 1:500), 2D6 (Mouse monoclonal antibody, cat# A246, Gentest, Woburn, Mass., 1:500), and 3A4/5 (Mouse monoclonal antibody, cat# A254, Gentest, Woburn, Mass., 1:500) for 30 min at room temperature. The immuno-complex was visualized by immunoglobulin enzyme bridge technique using Vector ABC-peroxidase kit (Vector Laboratories, Burlingame, Calif.). The enzyme substrate, 3,3′ diaminobenzidine tetrachloride was used, resulting in a brown reactant. Sections were then weakly counterstained with 0.1% hematoxylin. Concurrent sections were stained with antibodies to vimentin to assess antigen preservation. Appropriate negative (no primary antibody) and positive (kidney for 1A1/2 and 1B1, liver for 2B6, 2D6, and 3A4/5) controls were stained in parallel with each set of osteosarcoma blocks. The immunocytochemical stains were scored using a four tier scoring system (negative, low, medium, and highly positive).

Quantitative Immunofluorescence Staining for P450 3A4/5

De-paraffinized osteosarcoma sections were microwave-preheated in citric acid buffer to retrieve antigenicity. They were then permeabilized with 0.1% saponin for 10 min at room temperature, and treated with 3 washes of 0.02 M of glycine and 0.1% paraphenyldiamine (PPD) in TBS-Tween 20 to inactivate free formaldehyde molecules that might otherwise cause auto-fluorescence. Sections were blocked with blocking solution (5% goat normal serum and 1% fetal bovine serum in TBS-Tween 20) for 30 min at room temperature prior to being exposed to the P450 3A4/5 primary antibody (1:300) overnight at 4° C. Sections were then exposed to the labeled secondary antibody (ALEXA FLUOR 568 goat anti-mouse IgG, cat# A-1104, Molecular Probes, Eugene, Oreg., 1:100) for 30 min at room temperature. Cell nuclei were stained with Syto 16 (cat# S-7578, Molecular Probes, Eugene, Oreg., 1:4000) for 30 min at room temperature. The sections were mounted in anti-fading medium, and kept in the dark at 4° C. until examined.

Fluorescent Microscopy and Digital Imaging

Labeled sections were initially exited at λ=488 nm, and the fluorescing nuclei images were acquired at a 20× magnification by digital micrograph and ULTRAVIEW imaging software (Perkin-Elmer, Boston, Mass.). Consequently, sections were exited at λ=568 nm to acquire the fluorescing 3A4/5 enzyme image. Computer-generated composite images of nuclei and enzyme allowed visualization of cellular distribution. All images were acquired under identical conditions.

Statistical Method

All images were then partitioned into equal small areas (from approximately 10 cm² to as small as one pixel) to provide more homogenous regions, and pixel intensities were acquired from each of these areas. Nuclear-density-weighted average enzyme pixel intensity was then computed according to the following formula:

ΣWᵢJᵢ

where Jᵢ is the enzyme pixel intensity at the i^{th} area, and where Wᵢ is the weight of the i^{th} area whose value is given by the following formula:

W_{i=I}ᵢ/ΣIᵢ

where Iᵢ is the nuclei pixel intensity of i^{th} area, with the sum of Wᵢ normalized such as Σ Wᵢ=1 (Fisher L, Van Belle G. Biostatistics, a methodology for the health sciences. Applied probability and statistics. New York: John Wiley & Sons, Inc., 1993:329-330).

The null-hypothesis of no difference of weighted average enzyme intensity between metastatic biopsies and non-metastatic biopsies was tested using an exact permutation test at a significance level of 0.05.

### EXAMPLE 2

### Expression of P450s by Standard Immunocytochemistry

This example describes the analysis of P450 expression by immunocytochemistry. The results of experiment showed expression of 1A1/2, 1B1 and 3A4/5, while P450s 2B6 and 2D6 were not detectable. P450 1A1/2, although found to have high expression frequency and linked to sarcomas (Murray et al., J. Pathol., 171:49 [1993]) and many other types of cancer (Murray et al., Br. J. Cancer 77:1040 [1998]; Murray et al., J. Pathol., 177:147 [1995]) did not show any variation in the degree of staining. P450 1B1, an extra-hepatic enzyme known to be involved in the activation of a large number of procarcinogens (Shimada et al., Cancer Res., 56:2979 [1996]) with studies suggesting it has an endogenous role in some tumors (Taylor et al., Biochem. Soc. Trans., 24:328S [1996]), showed a high frequency of occurrence but very little variation in the degree of staining among the different osteosarcomas. P450 2B6, which is involved in the metabolism of cyclophosphamide (Roy et al., Drug Met. Disp. 27:655 [1999], and P450 2D6, which catalyzes the oxidative metabolism of a number of clinically important drugs, and has also been linked to hepatocellular carcinoma (Agundez et al., Pharmacogen 6:501 [1996]), did not appear to have a role in osteosarcomas in view of their total absence, unlike the rest of the P450s investigated.

Immunocytochemical analysis of osteosarcoma tissue micro array blocks representing all 18 biopsies using anti-P450s 1A1/2, 1B1, 2B6, 2D6, and 3A4 antibodies showed 83% (15/18) frequency for P450 1A1/2, 67% (12/18) frequency for P450 1B1, and 83% (15/20) frequency for P450 3A4/5. P450s 2B6 and 2D6 were shown to be totally absent in these osteosarcomas. P450 1A1/2 demonstrated medium immmunocytochemical staining with no variation among the different tumors. P450 1B1 also scored medium, but with some variation. P450 3A4 scored high staining with noticeable variation among the different cancers. All positive and negative immunocytochemical controls were as expected.

### EXAMPLE 3

### Expression of P450 3A4/5 by Quantitative Immunofluorescence (QICC)

This example describes the analysis of P450 expression by quantitative immunofluorescence. An uneven distribution of tumor cells and bony areas is likely to lead to problems in estimating the levels of the enzyme using standard techniques. Moreover, a non-weighted average of enzyme intensity leads to biased results due to the heterogeneity of the tumors cellular distribution. Therefore, a double-staining quantitative immunofluorescence technique (QICC) able to measure the levels of P450 enzymes in archival tumor sections was developed. Each image was partitioned into equally sized square areas, and nuclear density as well as enzyme intensity was estimated within each square area. The size and number of square areas were chosen in a way to render each sub-image more homogenous with respect to nuclear density. A weight average of enzyme intensity was then computed for each tissue section with weights given by the normalized nucleic intensity, assuming that it represents nuclear density. The weighted average intensity method allows targeting of richly cellular areas in the tumor by giving them a larger weight, and at the same time gives an appropriately smaller weight for less cellular more bony areas, thus, allowing a more reliable reading of the protein or enzyme in question.

Eighteen primary tumor sections (11 metastatic and 7 non-metastatic) were randomly chosen and were stained, scored in duplicates and averaged, as described previously. The experiment was done blinded with the different samples treated as unknowns. Then, these were identified and were placed in two groups based on occurrence of distant metastasis as shown in Table 1. Results demonstrated higher P450 3A4/5 levels in metastatic primary bone tumors with a weighted average mean intensity of 1228 compared to lower levels in non-metastatic primary bone tumors with a weighted average mean intensity of 536 (See FIG. 1).

Due to the small sample size of the tumor population, an exact permutation test was used instead of a two-tailed t-test to test the null hypothesis of no difference between the two groups of tumors. A permutation test is a non-parametric statistical analysis more appropriate test for small sample size populations.

An exact permutation test of the null hypothesis of no difference in the average P450 3A4/5 intensity between biopsies that eventually metastasized compared to ones that did not resulted in a p-value of 0.0004, a highly statistically significant difference illustrated in FIG. 1. Both groups were significantly different at an alpha level of 0.004 or greater.

TABLE 1
CYP3A4/5 levels in 18 primary osteosarcoma biopsies by QICC

| | Levels of 3A4/5 | | | |
|---|---|---|---|---|
| Subjects | (pixel intensity) | Distant Metastasis | Huvos Gr. | % necrosis |
| 1 | 1436 | Yes | II | 50 |
| 2 | 1495 | Yes | I | — |
| 3 | 1438 | Yes | II | 15 |
| 4 | 1052 | Yes | III | — |
| 5 | 1214 | Yes | IV | 100 |
| 6 | 1579 | Yes | IV | 99 |
| 7 | 1037 | Yes | III | 95 |
| 8 | 1470 | Yes. | — | — |
| 9 | 1296 | Yes | — | — |
| 10 | 0807 | Yes | II | 40 |
| 11 | 0696 | Yes | I | 15 |
| 12 | 0604 | No | — | 100 |
| 13 | 0531 | No | — | — |
| 14 | 0999 | No | III | 75 |
| 15 | 0532 | No | IV | 100 |
| 16 | 0438 | No | IV | 99 |
| 17 | 0172 | No | III | 90 |
| 18 | 0470 | No | — | 15 |

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for characterizing tumor tissue in a subject, comprising:
a) providing tumor tissue from a subject; and
b) detecting the presence or absence of P450 3A4/5 in said tumor tissue, thereby characterizing said tumor tissue.

2. The method of claim 1, wherein said tumor tissue is osteosarcoma tumor tissue.

3. The method of claim 1, wherein said tumor tissue is biopsy tissue.

4. The method of claim 1, wherein said detecting P450 3A 4/5 comprises detecting the presence of P450 3A 4/5 mRNA.

5. The method of claim 4, wherein said detecting the presence of P450 3A 4/5 mRNA comprises a detection assay selected from the group consisting of a Northern blot, in situ hybridization, reverse-transcriptase polymerase chain reaction, and microarray analysis.

6. The method of claim 1, wherein said detecting the presence of P450 3A 4/5 comprises detecting the presence of a P450 3A 4/5 polypeptide.

7. The method of claim 6, wherein said detecting the presence of a P450 3A 4/5 polypeptide comprises exposing said P450 3A 4/5 polypeptide to an antibody that specifically binds to P450 3A 4/5 and detecting the binding of said antibody to said P450 3A 4/5 polypeptide.

8. The method of claim 7, wherein said immunocytochemistry is quantitative immunocytochemistry.

9. The method of claim 1, wherein said characterizing comprises identifying the risk of said tumor tissue metastasizing based on said detecting the presence of P450 3A4/5.

10. A kit for characterizing cancer in a subject, comprising:
a) a reagent that specifically detects the presence of absence of expression of P450 3A 4/5; and
b) instructions for using said kit for characterizing cancer in said subject.

11. The kit of claim 10, wherein said reagent comprises an antibody that specifically binds to P450 3A 4/5.

12. The kit of claim 10, wherein said reagent comprises a nucleic acid probe that specifically binds to a P450 3A 4/5 mRNA.

13. The kit of claim 10, wherein said instructions comprise instructions required by the United States Food and Drug Administration for use in in vitro diagnostic products.

14. A method of screening compounds, comprising:
a) providing
i) an cell sample expressing P450 3A4/5; and
ii) one or more test compounds; and
b) contacting said sample with said test compound; and
c) detecting a change in viability of said cell sample in the presence of said test compound relative to the absence of said test compound.

15. The method of claim 14, wherein said test compound is a cancer chemotherapeutic.

16. The method of claim 14, wherein said test compound is a candidate cancer therapeutic.

17. The method of claim 14, wherein said cell sample is a cancerous cell sample.

18. The method of claim 17, wherein said cancerous cell sample is a osteosarcoma sample.

19. The method of claim 14, wherein said cell is in vitro.

20. The method of claim 14, wherein said cell is in vivo.
